(19) United States
(12) Patent Application Publication
(10) Pub. No.: US 2010/0310619 A1
(43) Pub. Date: Dec. 9, 2010

(54) Sonny's Miracle

(76) Inventor: Sulejman Selmani, Haledon, NJ (US)

Correspondence Address: Sulejman Selmani, 385 Mason Ave, Haledon, NJ, 07508, US

(21) Appl. No.: 12/411,368

(22) Filed: Jun. 3, 2009

(51) Int. Cl. A61K 9/06; A61K 36/28

(52) U.S. Cl. 424/402; 424/764

(57) ABSTRACT

An ointment made of sunflower oil and yellow candle (paraffin); the candles are dissolved in the heated sunflower oil container; the yellow candle and sunflower oil mixture turns into a cream which then the ointment is formed, while hot the ointment is pasted on a clean cloth by dipping the cloth into the heated cream mixture; the pasted cloth is kept chilled in the freezer until is ready to be applied; the ointment is applied chilled by applying the pasted cloth into the bum wound and is left on for 10 hours or less, after 10 hours the pasted cloth is replaced with another pasted cloth and the procedure is repeated until the ointment heals the wound, the chilled ointment immediately cools the bum wound and keeps it cool, removes the pain, heals the wound, does not leave wrinkles in the skin

### PURPOSE

Treats human body/skin from burns caused by fire, hot water or anything that can cause a burn on a human body/skin, it cures the burn wound area rapidly, it does not leave wrinkles on the body/skin and it also heals the pain.

### DESCRIPTION

This product is made out of yellow candles and sunflower oil in which are mixed and applied in a certain way.

### PROCEDURE

1. I heat up 2 liter sunflower cooking oil on a stainless steel cook pot, heat up the cooking oil until it boils and until the oil starts to bubble.

2. I add 1.8 kilogram yellow candles into the boiled sunflower cooking oil.

3. Leave the candles in the boiled sunflower cooking oil until the candles are completely melted and then the candles will turn into a cream; remove the cook pot with the cream mixture from the stove.

4. While hot the following procedure needs to be handled with care. Dip in a white clean cloth into the cook pot of the cream mix to paste the cream mixture into the cloth. Fold the cloth in a stick and dip in the stick into the cook pot to paste the cream mix into the cloth. Dip as many clothes as desired or until the cream in the cook pot is completely dissolved from the cloths.

Take the pasted cloth out of the cook pot, hang it in a place where it's safe for the cream mix to drip down and leave the cloth hanging until the cream mix stops dripping.

Once the dripping stops fold the cloth in half and place it in a box and continue to place additional clothes on top of the first cloth, fill up the box with the cream mix pasted cloths, and place the box with the pasted cloths in the freezer.

The box needs to be in the freezer and kept in the freezer until the clothes with the cream mix is requested to be applied into a burned skin.

5. If there is cream left in the cook pot, put it in a jar and place it in the freezer, and leave it in the freezer, the left over cream from the cook pot can be used as an ointment to treat burned skin, can be used in small burned affected areas of the skin. The pasted cloths are used for more severe burns.

6. The cloth with the pasted cream should be applied into a burned skin after is cooled in the fridge for at least 4-5 hours.

7. Leave the cloth on a burned affected skin area for 12 hours and replace the cloth after 12 hours with another cloth and leave it for another 12 hours. The other side of the cloth can also be used as long as the cream is not removed from the cloth, simply turn over the cloth on the other side and apply it on the affected burned skin area. After the first 12 hours the affected burned skin area will start to heal and show signs of improvements.

8. After applying step 7 procedure and after the cream mixture is applied and kept in the affected burned skin area for 48 hours, burn patients can remove the cloth and will notice fast results and feel better, the skin starts to heal, dries up and leaves no wrinkles in the skin, cures the burn wound and also heals the pain.

Advantages of the Invention: Helping burned patients to cure and recover from burn wounds rapidly with great results.

What is claimed is:

1. An ointment made of: sunflower oil and yellow candles (paraffin) mixed together in a certain way to form an ointment;

2. An ointment made of according to claim 1, wherein said the ointment is made out of two ingredients, sunflower oil and yellow candles the procedure includes: 2 liter of sunflower oil is heated in cook pot stainless steel container; the sunflower oil is heated until it starts to bubble.

3. An ointment made of according to claim 2, wherein said the oil is preheated the next step is to add 1.8 kilogram yellow candles into the container with the heated sunflower oil and mix the candles with the sunflower oil until the candles are completely dissolved.

4. An ointment made of according to claim 3, wherein said the candles are dissolved in heated sunflower oil, the ointment is now formed and the following will need to be performed to paste the ointment into the cloth. While hot the following procedure needs to be handled with care.
While the ointment is hot the cloth is dipped into the ointment with a stainless steel tongs, the cloth can be any size, as need it. hang the pasted cloth in a safe place where the ointment is safe to drip down and leave the cloth hanging until the ointment mix stops dripping.
paste as many clothes as desired with the ointment and fold the clothes in a box one cloth on top of the other.

5. An ointment made of according to claim 4, wherein said the ointment is pasted into a cloth and the pasted clothes are placed in a box, the boxes with pasted clothes are to be stored in the freezer and kept in the freezer until the ointment is ready to be applied in the burn wound.

6. An ointment made of according to claim 4, wherein said the ointment is pasted into clothes and for any additional ointment that is left in the cook pot container the ointment can be placed into a jar stored in a freezer which then the ointment can be used for treating minor burns.

7. An ointment made of according to claim 5, wherein said the pasted clothes are stored in the freezer, the next procedure is to apply the pasted chilled ointment into a burn wound, the chilled pasted cloth is applied into the burned affected area and is kept on the burn wound for 10 hours or less, after 10 hours or less, the cloth is replaced with another pasted cloth and kept on the same burn affected area for 10 hours or less.

8. An ointment made of according to claim 7, wherein said the pasted ointment needs to be applied chilled into a burn wound, the procedure in claim 7 is repeated until the skin starts to heal.

9. An ointment made of according to claim 7, wherein said to apply the pasted chilled ointment into a burn wound, after the chilled ointment is applied in the burn affected area it immediately cools the burn wound and keeps it cool, removes the pain and immediately starts to heal the burn wound and it does not leave any wrinkles on the skin.
